(19) United States
(12) Patent Application Publication
(10) Pub. No.: US 2026/0110565 A1
(43) Pub. Date: Apr. 23, 2026

(54) CALCULATION METHOD OF KARST UNDERGROUND RESERVOIR CAPACITY CURVE BASED ON HYDROGEOLOGICAL PARAMETERS

(71) Applicant: Yunnan Nengyang Water Conservancy and Hydropower Survey and Design Co., Ltd., Qujing, Yunnan (CN)

(72) Inventors: Dongsheng Wang, Qujing, Yunnan (CN); Chengbo Li, Qujing, Yunnan (CN); Guibao Ji, Qujing, Yunnan (CN); Huaqing Zhang, Qujing, Yunnan (CN)

(21) Appl. No.: 18/996,885

(22) PCT Filed: Sep. 27, 2023
(86) PCT No.: PCT/CN2023/121885

§ 371 Date: Jan. 17, 2025

(30) Foreign Application Priority Data
Aug. 25, 2023 (CN) 2023110766374

(51) Int. Cl. G01F 23/22; G01N 15/08; G01N 33/24; G01V 9/02

(52) U.S. Cl. CPC G01F 23/22; G01N 15/0826; G01N 33/246; G01V 9/02

(57) ABSTRACT

A calculation method of karst underground reservoir capacity curve based on hydrogeological parameters, including the following steps: hydrogeological exploration, determination of karst underground water reservoirs scope, determination of water storage space, determination of relevant hydrogeological parameters based on hydrogeological tests, and underground reservoir capacity analysis. The invention uses the unit water permeability and the borehole line karst rate that are very easily obtained through the borehole pressure (injection) water test and combines it with a small amount of water pumping test, thereby obtaining a correlation function relationship through regression analysis between the specific yield and the karst rate that are difficult to obtain and the unit water permeability or the permeability coefficient; the underground storage capacity at the corresponding elevation is calculated according to the specific yield or the karst rate and the underground storage capacity curve is obtained, which can improve accuracy and reduce errors.

### TECHNICAL FIELD

The invention relates to the technical field of karst underground reservoir capacity calculation, and in particular to a calculation method of karst underground reservoir capacity curve based on hydrogeological parameters.

### BACKGROUND ART

Underground storage capacity refers to the pores, cracks and karst cave aquifer cavities below the ground that have water storage value. There is no systematic literature discussing the underground reservoir storage capacity at home and abroad, and it has always been a blank area in theoretical research. The lag in theoretical research has restricted the rational development and utilization of aquifers.

The study of the storage capacity of karst caves relative to pore and fissure-type underground reservoirs, especially the underground storage capacity curve, is of great significance to the effective implementation of reservoir area planning and dispatching schemes. Because the water storage medium is extremely uneven, the analysis is relatively difficult. The following methods are commonly used to calculate the storage capacity of underground reservoirs:

### 1. Groundwater Balance Method

Based on the measured runoff of the karst underground river, the annual distribution analysis of the designed annual runoff (P=75%) was carried out, and the runoff in April, the driest period, was taken as the stable runoff, and the underground storage capacity was not less than the sum of the stable runoff and the seasonally increased runoff in the dry season (January to May) as the karst underground storage capacity.

### 2. Plotnikov Method for Groundwater Runoff

Plotnikov classified groundwater reserves into:

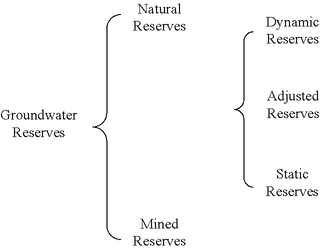

The dynamic reserve is the volume of groundwater flowing through a certain water-passing section of the aquifer under natural conditions per unit time; the regulating reserve is the volume of water stored in the aquifer between the highest and lowest water levels under natural conditions; the static reserve is the part of the water storage volume that needs to be extracted by wells; the underground storage capacity is the regulating reserve of the volume of water stored in the aquifer between the highest and lowest water levels under natural conditions, which is the underground storage capacity.

### 3. Other Methods

There are also the following methods for calculating underground reservoir capacity based on the few domestic and foreign literature:
1. Specific yield method: for porous and fractured underground storage capacity, specific yield obeys different probability distributions (normal distribution) in space. Based on the determined specific yield distribution, the spatial overlay function of ArcGIS software is used to overlay and analyze the specific yield zoning map of the phreatic aquifer in the study area with the average phreatic water level and surface elevation of a certain year, and the drainage storage capacity of the phreatic aquifer in that year is calculated. Then the whole area is divided into rectangles, and the buried depth value of each cell is calculated in combination with the surface elevation and the average phreatic water level of that year. According to the needs of actual analysis, the Monte Carlo method is used for multiple random simulations (fuzzy method). Each simulation generates a number of specific yield values that meet a certain distribution, and obtains the corresponding multiple drainage storage capacity values.
2. Water content method: the water content of the karst body is affected by the porosity and water absorption of the rock mass and is determined by the weighted average of the water content of the cross section. The volume of the underground system (underground storage capacity) is determined by the product of the underground rock volume and the average water content.
3. Mathematical model method of tracer detection: for the complex karst seepage field of solution crevice type, it can be generalized into two karst hydrogeological models: pipeline-solution crevice dual parallel flow field and single pipeline series pool flow field. The calculation is based on the average groundwater flow and the total mass of the tracer, the average concentration or peak concentration, and the time required for the recovery to reach half or all.
4. Water storage space geometric morphology generalization method:
the volume of karst caves was calculated based on actual measurements.
5. Water tank model simulation method (inversion method): that is, in the evaluation of underground storage capacity, it is usually obtained by inverting the original data of the reservoir operation and scheduling records after the underground reservoir is filled with water. By releasing water (water release test) from the underground river retaining space and water storage space (water tank), the water flowing out of the water tank is the underground storage capacity. The storage and release of the water tank can be linear or nonlinear, usually linear.

In summary, the existing research data on karst underground storage capacity are reviewed, and it is mainly demonstrated from a macroscopic perspective that no matter which method is used, there are certain errors and precisions, and only a total underground storage capacity result can be obtained. It is difficult to obtain its specific yield, karst rate, and water content, and it is even more difficult to measure a large number of karst fissures. The tracer detection method has tracer adsorption reduction and it is difficult to estimate the non-water-filled cavity. The water tank simulation method is carried out after the reservoir is filled with water, and has no guiding role in the preliminary survey and design. It is even worse for the guidance of underground reservoir water storage, scheduling and flood control, and it is difficult to understand the underground storage capacity characteristics at different elevations.

### SUMMARY OF THE INVENTION

In order to guide the survey, design, water storage, scheduling, and flood control of underground reservoirs, it is necessary to understand more about the water storage capacity under different water storage elevations, so as to determine the corresponding water storage and water use plans according to different elevations and formulate corresponding flood control measures. It is necessary not only to obtain the total reservoir capacity, but also to understand the reservoir capacity characteristics under different water storage level conditions.

The technical solution provided by the invention is:
a calculation method of karst underground reservoir capacity curve based on hydrogeological parameters, including the following steps:
S1. hydrogeological exploration: conducting hydrogeological exploration of the target karst groundwater reservoir, including exploring the karst conditions of the dam site and reservoir area, and the distribution of karst groundwater;
S2. determination of karst underground water reservoirs scope: according to the hydrogeological conditions of the underground water reservoir basin of the target underground water reservoir, as well as the groundwater overflow elevation, groundwater springs and hydrogeological borehole data at the karst watershed, obtaining the groundwater level in the dry season and drawing the water level contour map accordingly to determine the boundary and bottom limit conditions of the underground water reservoir capacity;
S3. determination of water storage space: through ground geological mapping, calculating the karst fissure rate at multiple points, further surveying the karst caves between the highest water storage level and the lowest groundwater level, focusing on finding out the volume and elevation distribution of the karst caves that can be explored, and calculating the volume distribution of water storage rock bodies and karst caves that can be explored at different elevations above the lowest groundwater level;
S4. determination of relevant hydrogeological parameters based on hydrogeological tests: arranging multiple groups of exploration boreholes and pumping test holes in the catchment area of the target underground reservoir, and conducting a large number of borehole pressure/water injection tests to obtain hydrogeological parameters, including borehole void encounter rate and unit water permeability, and analyzing the drainage volume and specific yield of the cone of depression, as well as the specific yield of each elevation of the underground reservoir basin according to the depression curve obtained from the pumping test;
S5. underground reservoir capacity analysis: based on the borehole data, statistically analyzing the borehole line karst rate of each borehole, including the filled and unfilled types, and the unit water permeability according to the elevation distribution, and using the relevant section length for weighted average to calculate the borehole line karst rate and unit water permeability of each comprehensive borehole at each elevation;
according to the regression relationship between karst specific yield and comprehensive borehole line karst rate and unit water permeability μ=0.525n+0.088q, calculating the specific yield of each elevation involved in the underground reservoir capacity;
for underground storage capacity, the underground storage capacity should be the volume of all karst caves that can be filled with water in the underground karst body, which is determined by the product of the karst caves V_{cave} that can be explored and measured plus the remaining soluble rock volume V_{solution crack} after deducting the karst caves that can be explored and measured and the above karst specific yield V_{solution crack}, then the underground karst storage capacity is V=V_{cave}+V_{solution crack},
wherein V_{cave} is an unfilled karst cave of no less than 300 m³; V_{solution crack}=Vₜₒₜₐₗ−V_{cave}; V_{solution crack}=μv_{solution crack}.

The advantages of the invention compared with the prior art are as follows: the invention uses the unit water permeability and the borehole line karst rate that are very easily obtained through the borehole pressure (injection) water test and combines it with a small amount of water pumping test, thereby obtaining a correlation function relationship through regression analysis between the specific yield and the karst rate that are difficult to obtain and the unit water permeability or the permeability coefficient; the underground storage capacity at the corresponding elevation is calculated according to the specific yield or the karst rate and the underground storage capacity curve is obtained, which can improve accuracy and reduce errors, and can play a guiding role in the water storage, scheduling, and flood control of underground reservoirs.

### BRIEF DESCRIPTION OF THE DRAWINGS

FIG. 1 is an annual distribution diagram of the underground river P=75% of the designed annual runoff in the method according to the invention.

FIG. 2 is a schematic diagram of the hydrogeology and exploration borehole arrangement of the stone forest underground reservoir in the method according to the invention.

FIG. 3 is a cross-sectional view of the pumping hole in the method according to the invention.

FIG. 4 is a graph showing the underground reservoir capacity in the method according to the invention.

### SPECIFIC EMBODIMENT OF THE INVENTION

In order to make the objectives, technical solutions, and advantages of the embodiments of the invention clearer, the technical solutions in the embodiments of the invention will be described clearly and completely hereinafter with reference to the drawings in the embodiments of the invention. Obviously, the described embodiments are part of the embodiments of the invention, rather than all of the embodiments. The components of the embodiments of the invention generally described and illustrated in the drawings herein may be arranged and designed in various different configurations.

### Embodiment

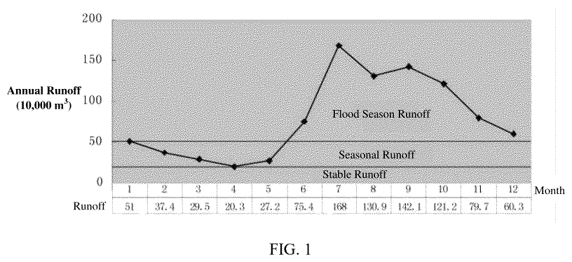

This Embodiment takes stone forest underground reservoir as an example. Stone forest underground reservoir is located in the stone forest karst area of Yunnan Plateau at the source of Bajiang River, a tributary of Nanpanjiang River in the middle and upper reaches of Xijiang River system in the Pearl River Basin, wherein karst is very developed. The total underground and surface runoff area is 26.63 km², and the average groundwater runoff for many years is 12.98 million m³ for P=50%, 9.41 million m³ for P=75%, and the flow rate is 0.044-4.35 m³/s, as shown in FIG. 1.

The reservoir consists of two ground reservoir basins (Xiangshuidong karst funnel reservoir basin and Qingshikan karst basin reservoir basin) and one underground reservoir basin. The main buildings are composed of the Qingshikan karst basin reservoir basin dam, inter-reservoir connecting tunnels, water transfer culverts, water collection corridors, underground river dredging tunnels, etc. The inlet bottom plate elevation of the water transfer culvert (high culvert) is 1851.50 m. The bottom width of the underground river dredging tunnel gate is 1.6 m and the bottom plate elevation is 1822.50 m.

The hydrogeological conditions of underground reservoirs in karst areas are relatively complex. The underground reservoir basin is composed of underground rivers, caves, rock karst gaps, and fissures. To intercept and fully utilize the underground karst caves through engineering measures to form underground reservoirs and regulate water, it is necessary to study the underground reservoir capacity characteristics, focusing on the reservoir capacity characteristics at different elevations.

With reference to FIGS. 1-4, the method of the Embodiment includes the following steps:

### S1. Brief Introduction of Hydrogeology and Exploration

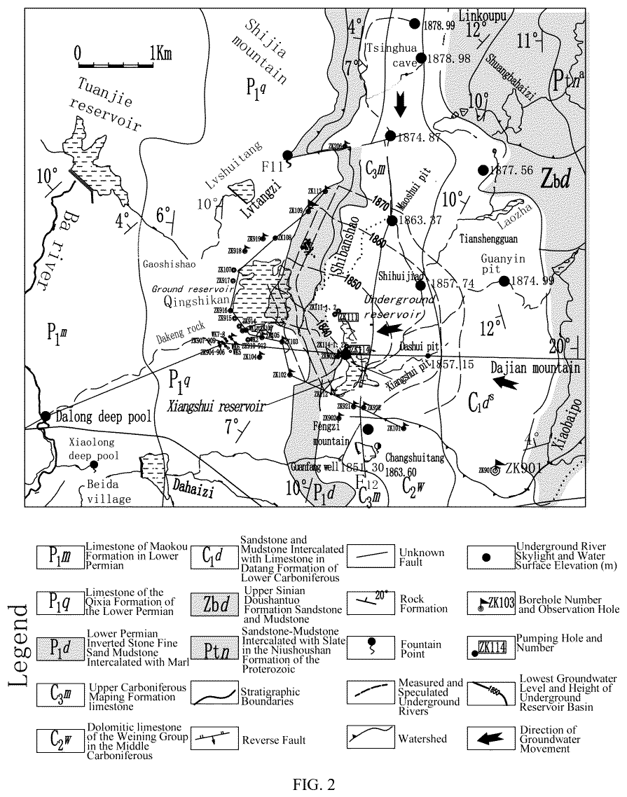

The stone forest underground reservoir is located in the karst platform of eastern Yunnan. Since 1966, an underground hydrological observation station has been established, and project demonstration, feasibility study, preliminary design and construction have been carried out successively. The karst strata involved in the stone forest underground reservoir are the limestones of the Qixia Formation of the Carboniferous and the Lower Permian System. The dam site is located in the sandstone and mudstone intercalated with mud limestone of the Daoshitou Formation of the Lower Permian System between two layers of limestone. As shown in FIG. 2, the underground reservoir area is formed by intercepting and blocking underground rivers, karst caves and karst fissures, raising the groundwater level, building dams in the hub area and storing water in the upstream karst depression to form a surface reservoir to achieve gravity specific yield.

According to the karst development characteristics of stone forest underground reservoir and the layout of engineering buildings, a total of 47 exploration boreholes were completed, with a borehole footage of 3,319 m and 567 sections of water pressure (injection) tests; 18 boreholes in the underground reservoir basin (including 6 pumping holes and monitoring holes) were involved. In order to find out the karst conditions of the dam site and reservoir area, and the distribution of karst groundwater, the boreholes were mainly arranged in the dam site and the curtain anti-seepage axis. At the same time, according to the karst characteristics of the reservoir area, two pumping test areas were arranged in the karst developed and less developed areas of the reservoir area. Each pumping test area was arranged with a pumping hole, and two groundwater level observation holes were arranged about 15-20 m and 50 m outside the pumping hole.

### S2. Determination of Karst Underground Water Reservoirs Scope:

according to the hydrogeological conditions of the underground water reservoir basin of the target underground water reservoir, as well as the groundwater overflow elevation, groundwater springs and hydrogeological borehole data at the karst watershed, obtaining the groundwater level in the dry season and drawing the water level contour map accordingly to determine the boundary and bottom limit conditions of the underground water reservoir capacity;

### S3. Determination of Water Storage Space:

through ground geological mapping, calculating the karst fissure rate at multiple points, further surveying the karst caves between the highest water storage level and the lowest groundwater level, focusing on finding out the volume and elevation distribution of the karst caves that can be explored, and calculating the volume distribution of water storage rock bodies and karst caves that can be explored at different elevations above the lowest groundwater level;

### S4. Determination of Relevant Hydrogeological Parameters Based on Hydrogeological Tests:

in order to analyze the underground storage capacity curve, it is important to obtain the specific yield of the relevant elevation, and the specific yield involves many relevant hydrogeological parameters. Since a certain scale of exploration boreholes and pumping test holes are arranged in the catchment area of the underground reservoir, a large number of borehole pressure (injection) water tests have been carried out, which makes it easy to obtain relevant rock unit permeability and borehole line karst rate and other hydrogeological parameters.

### 1. Easily Accessible Hydrogeological Parameters

(1) The borehole line karst rate (n: %) is the ratio of the total length of the karst caves seen in the borehole l (m) to the borehole footage of this section L (m):

$$n = \frac{l}{L} .$$

(2) Unit water permeability (q: lu): the amount of water injected (Q: l/min) under unit pressure (P: MPa) and unit test section length (L: m):

$$q = \frac{Q}{PL} .$$

### (3) Permeability Coefficient

it is mainly obtained from the two pumping test holes ZK114 and ZK111. There are many methods for calculating the permeability coefficient. According to the “Specific yield Hydrogeological Survey Code GB50027-2001” and “Hydropower and Water Conservancy Project Drilling Pumping Test Code DL/T5213-2005”, there are mainly specific yield specification formulas, Qiu Buyi, Babushkin, Skabaranovich and other formulas. The results obtained by analysis are 8.355-23.022 times different from each other. The average value of various methods is close to the Babushkin formula, that is:

$$K = 0.366 \frac{Q}{ls} lg \frac{0.66 l}{r}$$

in the formula: K is the permeability coefficient m/d, Q is the water inflow m³/d, l is the filter length (m), s is the drawdown (m), and r is the borehole radius (m).

### 2. Depression Curve and Drainage Volume

(1) Pumping test and related results are shown in Table 1;
(2) They are calculated according to the Kusakin influence radius formula and the Weber specific yield formula:

Kusakin influence radius formula: R=2s\sqrt{KH};
in the formula: s is the influence radius of the cone of depression (m), K is the permeability coefficient m/d, and H is the thickness of the phreatic aquifer (m); the calculated impact radius is 26 m-206 m, which is somewhat different from the measured data.

TABLE 1
Comprehensive Results of Pumping Test

| Content | Unit | ZK114 | | | | ZK111 | | | |
|---|---|---|---|---|---|---|---|---|---|
| Static Water | m | 1839.55 | 1839.55 | 1839.55 | 1839.55 | 1852.22 | 1852.22 | 1852.22 | 1852.22 |
| Level | | | | | | | | | |
| Dynamic Water | m | 1835.99 | 1832.25 | 1821.45 | 1803.86 | 1849.02 | 1842.07 | 1840.13 | 1837.23 |
| Level | | | | | | | | | |
| ZK114 Hole | m | 3.56 | 7.3 | 18.1 | 35.69 | 3.2 | 10.15 | 12.09 | 14.99 |
| Drawdown s | | | | | | | | | |
| ZK114-2 Hole | m | 0 | 2.67 | 6.21 | 11.12 | 0 | 0 | 0 | 0.02 |
| Drawdown s1 | | | | | | | | | |
| ZK114-1 Hole | m | 0 | 0 | 0 | 0.058 | 0 | 0 | 0 | 0 |
| Drawdown s2 | | | | | | | | | |
| Hole Distance | m | 16.16 | 16.16 | 16.16 | 16.16 | 24.76 | 24.76 | 24.76 | 24.76 |
| from ZK114-2 to | | | | | | | | | |
| 114 | | | | | | | | | |
| Hole Distance | m | 51.76 | 51.76 | 51.76 | 51.76 | 63.53 | 63.53 | 63.53 | 63.53 |
| from ZK114-1to | | | | | | | | | |
| 114 | | | | | | | | | |
| Borehole | m | 0.065 | 0.065 | 0.065 | 0.065 | 0.065 | 0.065 | 0.065 | 0.065 |
| Diameter r | | | | | | | | | |
| Duration t | min | 300 | 620 | 6600 | 19770 | 95 | 230 | 4365 | 7020 |
| Volume of Water | m³ | 17.3 | 39.36 | 630.3 | 2040 | 1 | 2.7 | 59.2 | 108.5 |
| Discharged W | | | | | | | | | |
| Flow Q | m/d | 84.48 | 99.36 | 141.89 | 148.58 | 15.64 | 17.86 | 19.67 | 22.25 |
| Specific Field Q | l/m · s | 45.3 | 37.45 | 34.25 | 25.7 | 4.89 | 1.76 | 1.63 | 1.48 |
| Filter Length | m | 21.7 | 21.7 | 21.7 | 21.7 | 14 | 14 | 14 | 14 |
| Aquifer | m | 51.74 | 51.74 | 51.74 | 51.74 | 63.53 | 63.53 | 63.53 | 63.53 |
| Thickness H | | | | | | | | | |
| Permeability | m/d | 0.938 | 0.538 | 0.310 | 0.165 | 0.275 | 0.099 | 0.092 | 0.084 |
| Coefficient K | | | | | | | | | |

According to Weber formula:

$$R = 3 \sqrt{\frac{KHt}{μ}}$$

is transformed into:

$$μ = 9 \frac{K H t}{{R}^{2}} .$$

In the formula: μ is the specific yield (%), t is the pumping time (d), and the specific yield calculated by using the influence radius of the Kusakin formula is 0-5%, with an average of 1.98%, which is difficult to correlate with the karst development at various elevations of the underground reservoir basin and the specific yield. The specific yield calculated according to the measured influence radius is 0.4-4989%, which is a large difference.

### (3) Drainage Volume of the Cone of Depression

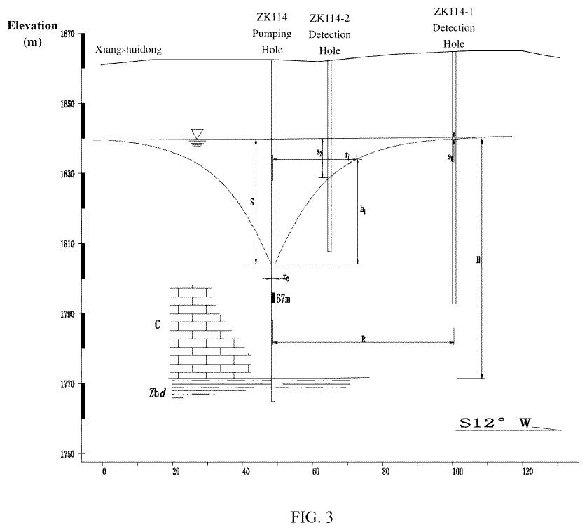

According to the observation results of groundwater drawdown curves of ZK111 and 114 holes (as shown in FIG. 3), fitting is performed:
assuming: R is the maximum radius of the cone of depression (m), s is the maximum groundwater drawdown (m), rᵢ is the distance from the pumping hole axis (m), hᵢ is the water level (m) at rᵢ above the maximum drawdown elevation of the pumping hole, and K is the permeability coefficient m/d;
according to the observation of the depression curve, rᵢ is related to hᵢ, K and s, and has an obvious exponential function relationship with hᵢ: assuming rᵢ to be a composite function of

$${h}_{i}^{α} ,$$

K^{β}, and s^{γ}, and performing trial calculations based on

$${h}_{i}^{α} ,$$

K^{β}, and s^{γ}, when α, β, and γ are 3.5, 0.46, and 1.02, respectively, the correlation coefficients between the calculated value and the measured value are 0.99, 0.95, and 0.97, respectively; the depression curve equation is:

$${r}_{i} = ξ {s}^{1.02} {K}^{0.46} {h}_{i}^{3.5} ;$$

due to the small sample size, the regression coefficient is 0.96, which is highly correlated.

in the formula, the coefficient ξ is adjusted according to the corresponding depression curve shape, and its value is 0.49-1.16; s and K are fixed values, so the comprehensive coefficient is set to be m=ξs^{1.02}K^{0.46}.
the volume of the body rotating around the h (y) axis of the depression curve is:

$$V = π {\int }_{0}^{{h}_{max}} {[ {( \frac{h}{m} )}^{3.5} ]}^{2} d h = \frac{π}{{m}^{7}} \frac{{h}^{8}}{8} {\vert}_{0}^{{h}_{max}} = \frac{π {h}_{max}^{8}}{8 {m}^{7}}$$

for 114 hole ξ=1.04-1.16, 111 hole ξ=0.49-0.75, the calculation results are shown in Table 2.

### 3. Specific Yield Correlation Analysis

the specific yield μ/% is determined by the ratio of the amount of water discharged by the cone of depression Q/m³ to the volume of the cone of depression V/m³, that is:

$$μ = \frac{Q}{V} .$$

### (2) Correlation Analysis of Specific Yield at Each Elevation of Underground Reservoir Basin

In order to obtain the specific yield at different elevations of the underground reservoir basin, firstly, making full use of the relevant hydrogeological parameters obtained from the pumping test wells for regression analysis; for two relevant parameters, performing a univariate linear regression; if two or more parameters are involved, using a binary or multivariate regression analysis.

TABLE 2
Calculation Results of Cone of Depression

| | | | | | | | | Filling the | Filling the | |
|---|---|---|---|---|---|---|---|---|---|---|
| | | | | | | | | Borehole | Borehole | |
| | | | | | Volume of | Volume | | Void | Void | |
| | Permeability | | Maximum | Maximum | Cone of | of Water | Specific | Encounter | Encounter | Unit Water |
| Borehole | Coefficient | Comprehensive | Drawdown | Radius | Depression | Discharged | Yield μs | Rate (%) | Rate (%) | Permeability |
| Number | K (m/d) | Coefficient m | hmax (m) | (m) | (m³) | (m³) | (%) | as 0.51 | as 1 | (lu) |
| ZK114 | 0.938 | 1.72 | 3.56 | 13.423 | 227.5 | 17.3 | 7.60 | 2.2 | 4.4 | 38.78 |
| | 0.538 | 3.01 | 7.3 | 23.636 | 1414.7 | 39.4 | 2.78 | 1.1 | 2.2 | 20.33 |
| | 0.31 | 6.88 | 18.1 | 31.603 | 6199.7 | 617.2 | 9.96 | 1.3 | 2.5 | 14.24 |
| | 0.162 | 9.25 | 26.58 | 43.305 | 16885.2 | 630.3 | 3.73 | 3.7 | 7.1 | 20.98 |
| | 0.162 | 11.63 | 35.69 | 54.940 | 35923.1 | 2040.0 | 5.68 | 3.4 | 6.2 | 18.18 |
| ZK111 | 0.275 | 1.88 | 3.2 | 6.678 | 52.0 | 1.0 | 1.98 | 0.5 | 1 | 30.70 |
| | 0.137 | 4.73 | 10.15 | 15.270 | 835.1 | 2.7 | 0.32 | 0.3 | 0.5 | 21.70 |
| | 0.092 | 4.91 | 12.09 | 24.952 | 2605.6 | 59.2 | 2.27 | 2.4 | 3.4 | 16.35 |
| | 0.082 | 5.31 | 14.99 | 38.740 | 8410.3 | 108.5 | 1.29 | 2.4 | 3.4 | 13.14 |

(2.1) univariate linear regression uses a linear function formula: y=a+bx (linear function) or y=10^{a}x^{b} (exponential function);
then

$$b = \frac{{{\sum }_{i = 1}}^{n} ( {x}_{i} - x ) ( {y}_{i} - y )}{{\sum }_{i = 1}^{n} {( {x}_{i} - \overline{x} )}^{2}} ,$$

a=y̅−bx̅, wherein: x̅ and y̅ are average values; the correlation coefficient is:

$$r = \frac{{{\sum }_{i = 1}}^{n} ( {x}_{i} - x ) ( {y}_{i} - \overline{y} )}{\sqrt{{\sum }_{i = 1}^{n} {( {x}_{i} - x )}^{2} {\sum }_{i = 1}^{n} {( {y}_{i} - \overline{y} )}^{2}}} ;$$

(2.2) assuming that the multiple linear regression is:

$$X = [ \begin{matrix}1 & {x}_{11} & … & {x}_{1 n} \\ 1 & {x}_{21} & … & {x}_{2 n} \\ ⋮ & ⋮ & ⋮ & ⋮ \\ 1 & {x}_{n 1} & … & {x}_{nn}\end{matrix} ] , Y = [ \begin{matrix}{y}_{1} \\ {y}_{2} \\ ⋮ \\ {y}_{n}\end{matrix} ]$$

then the regression coefficient β=(X^{T}X)⁻¹X^{T}Y, and the regression equation y=β₀+β₁x₁+β₂x₂+ . . . +βₙxₙ (linear function), or

$$y = \begin{matrix}{10}^{{β}_{0}} & {x}_{1}^{{β}_{1}} & {x}_{2}^{{β}_{2}} & … & {x}_{n}^{{β}_{n}}\end{matrix}$$

(exponential function), and the multiple correlation coefficient is:

$${r}_{xy} = \frac{{{\sum }_{i = 1}}^{n} ( {y}_{i} - \overline{y} ) ( {\hat{y}}_{i} - \overline{y} )}{\sqrt{{\sum }_{i = 1}^{n} {( {y}_{i} - \overline{y} )}^{2} {\sum }_{i = 1}^{n} {( {\hat{y}}_{i} - \overline{y} )}^{2}}}$$

(2.3) the correlation coefficients r and r_{xy} are dimensionless values, and the range of r and r_{xy} is [−1,1]; when r=1, it is a completely positive correlation, and when r=−1, it is a completely negative correlation, wherein:
when |r| or |r_{xy}|>0.8, there is a high or significant correlation; when 0.5<|r| or |r_{xy}|≤0.8, there is a moderate correlation; when 0.3<|r| or |r_{xy}|≤0.5, there is a low correlation; when |r| or |r_{xy}|≤0.3, there is no linear correlation;
(2.4) conducting a univariate linear correlation analysis between the borehole specific yield μₛ/% and the average borehole line karst rate n/%, permeability coefficient K/m/d, and unit water permeability q/lu in the corresponding drop depth according to the existing data; the results are shown in Table 3.

TABLE 3
Correlation Analysis Results of Specific yield, Karst
Rate of Borehole Line and Unit Water Permeability

| | Specific Yield (μ) | | Permeability Coefficient (K) | |
|---|---|---|---|---|
| | | Regression | | Regression |
| Items | Regression Equation | Coefficient r | Regression Equation | Coefficient r |
| Permeability | μ = 5.007K + 2.031 | 0.68-Moderate | | |
| Coefficient (K) | | Correlation | | |
| Borehole Line Karst | μ = 1.043n + 0.359 | 0.63-Moderate | q = 0.481n + 2.988 | 0.10-No |
| Rate (n) | | Correlation | | Correlation |
| Unit Water | μ = 0.157q + 0.066 | 0.62-Moderate | q = 23.33K + 15.13 | 0.82-High |
| Permeability q | | Correlation | | Correlation |

In the borehole pressure/water injection test, the more developed the karst is, the greater its unit permeability is; usually, for unfilled karst caves, it is impossible to conduct pressure/water injection tests when they have a certain width; during the pressure/water injection test, it is difficult to conduct statistics on the encounter rate of borehole caves for parts with smaller unit permeability; therefore, to examine the karst development intensity and karst rate of a part, it is necessary to combine the karst rate and unit permeability of the borehole line for simultaneous analysis; the filling material of the filled karst caves is mainly gravel clay or gravel sandy clay; through the physical property test of the filling soil, the average porosity of the filling soil is 0.51; therefore, the porosity of the filled karst caves is calculated as 51%; the comprehensive borehole line karst rate should be determined by adding 51% of the filled borehole line karst rate to 100% of the unfilled karst cave rate.

(2.5) according to ZK114 and ZK111 borehole karst specific yield and the comprehensive borehole line karst rate and unit water permeability are subjected to binary regression, and the relationship is: μ′=−4.18+1.32n+0.21q, the multiple correlation coefficient is 0.89, which is highly correlated; after canceling the coefficient −4.18, analyzing the specific yield μ′ calculated by binary regression and the actual specific yield μₛ again by univariate regression to obtain the comparison expression: μ=0.525n+0.088q, the correlation coefficient is 0.94, which is highly correlated. The results are shown in Table 4.

TABLE 4
Specific Yield Calculation Results

| | | | Specific Yield | |
|---|---|---|---|---|
| Borehole Void | Unit Water | Measured | Calculated by | Final Calculated |
| Encounter Rate | Permeability | Specific Yield | Binary Regression | Specific Yield μ |
| n (%) | q (lu) | μs (%) | μ′ (%) | (%) |
| 4.4 | 38.78 | 7.73 | 9.92 | 5.72 |
| 2.2 | 20.33 | 2.8 | 3.07 | 2.94 |
| 3.7 | 20.98 | 3.73 | 5.18 | 3.79 |
| 4.4 | 18.18 | 5.68 | 5.51 | 3.91 |
| 0.5 | 30.7 | 1.98 | 3.05 | 2.96 |
| 3.4 | 16.35 | 2.27 | 3.8 | 3.22 |
| 3.4 | 13.14 | 1.29 | 3.11 | 2.94 |

### S5. Underground Storage Capacity Curve Analysis

The karst bodies in the area involve the Qixia Formation limestone of the Carboniferous and Lower Permian. After statistical analysis, the average karst rate and weighted average unit permeability of the Carboniferous and Permian limestone drilling lines in the area are shown in Table 5.

Based on the borehole data, statistically analyzing the borehole line karst rate of each borehole, including the filled and unfilled types, and the unit water permeability according to the elevation distribution, and using the relevant section length for weighted average to calculate the borehole line karst rate and unit water permeability of each comprehensive borehole at each elevation;

The underground storage capacity only involves the Carboniferous limestone above the lowest groundwater level, so only this part of the rock mass is considered in the statistics. Since the karst caves of limestone with a unit permeability of highly permeable (q≥1000 lu) are often opened to more than 3 mm, these caves are easy to measure with the increase of the width of the karst caves. However, during the pressure (injection) water test, the test section above the groundwater level usually cannot raise the test water level to the top of the test section. Its unit permeability is as high as tens of thousands of lu, and even accurate results cannot be obtained. The water content obtained from this is mostly greater than 100%, so the data of the highly permeable section are eliminated. According to the regression relationship between the karst specific yield and the comprehensive borehole line karst rate and unit permeability μ=0.525n+0.088q, the calculation results of the specific yield at each elevation involved in the underground storage capacity are shown in Table 6.

TABLE 5
Average Karst Rate of Borehole Lines and Weighted Average Permeability Length

| | Carboniferous Limestone | | | | | Permian Limestone | | | | |
|---|---|---|---|---|---|---|---|---|---|---|
| | | Karst | Karst | | | | Karst | | | |
| | | Rate of | Rate of | | | | Rate of | Karst | | |
| | | Borehole | Borehole | | | | Borehole | Rate of | | |
| | | Filled | Unfilled | | | | Filled | Borehole | | |
| | | Type | Type | | Unit Water | | Type | Unfilled | | Unit Water |
| | Number | Borehole | Borehole | Unit Water | Permeability | Number | Borehole | Type | Unit Water | Permeability |
| | of | Line | Line | Permeability | Eliminating ≥1000 | of | Line | Borehole | Permeability | Eliminating ≥1000 |
| | Samples | (%) | (%) | (lu) | lu (lu) | Samples | (%) | Line (%) | (lu) | lu (lu) |
| Above- | 54 | 4.48 | 0.00 | 5982.45 | 36.38 | 103 | 5.35 | 0.88 | 229.79 | 112.07 |
| Water | | | | | | | | | | |
| Part | | | | | | | | | | |
| Below- | 270 | 2.68 | 0.05 | 30.31 | 19.25 | 37 | 1.21 | 0.00 | 41.86 | 13.17 |
| Water | | | | | | | | | | |
| Part | | | | | | | | | | |
| Total | 324 | 2.85 | 0.05 | 1656.06 | 12.37 | 140 | 4.55 | 0.71 | 194.59 | 22.79 |

TABLE 6
Calculation Results of Karst Specific Yield at Elevation

| | | Borehole Line Karst Rate % | | | | | | |
|---|---|---|---|---|---|---|---|---|
| | | | | | Filled Type | | | |
| | | Filled | Unfilled | | Void | Unit Water | | |
| | | Type | Type | | Encounter | Permeability lu | | Specific |
| Bottom | Top | Cavity | Cavity | Total | Rate (The | Unit Water | Total | Yield |
| Elevation | Elevation | Length | Length | Length | Porosity is | Permeability | Length | μ |
| (m) | (m) | (m) | (m) | (m) | 51%) | (u) | (m) | (%) |
| 1875 | 1880 | | | 14.85 | 0 | 32.56 | 14.85 | 2.19 |
| 1870 | 1875 | 3.73 | 0 | 30 | 9.51 | 22.72 | 20 | 9.37 |
| 1865 | 1870 | 2.67 | 0 | 34.12 | 3.99 | 50.47 | 34.12 | 7.17 |
| 1860 | 1865 | 2.29 | 0 | 40 | 2.92 | 15.02 | 40 | 4.54 |
| 1855 | 1860 | 0.05 | 0 | 44.85 | 0.06 | 70.36 | 44.85 | 2.73 |
| 1850 | 1855 | 0.25 | 0 | 35 | 0.32 | 54.32 | 40 | 4.3 |
| 1845 | 1850 | 0.05 | 0 | 15 | 0.09 | 33.61 | 30 | 5.42 |
| 1840 | 1845 | 0 | 0 | 15 | 0 | 20.76 | 15 | 3.34 |
| 1835 | 1840 | 0 | 0 | 10 | 0 | 17.86 | 20 | 2.28 |
| 1830 | 1835 | 0.45 | 0 | 10 | 2.30 | 11.27 | 10 | 4.35 |
| 1825 | 1830 | 0 | 0 | 5 | 0 | 5.38 | 10 | 1.34 |
| 1820 | 1825 | 0 | 0 | 5 | 0 | 6.28 | 5 | 0.72 |

| | | | | | | | |
|---|---|---|---|---|---|---|---|
| Total | 9.49 | 0 | 283.82 | 1.71 | 36.87 | 283.82 | 4.14 |

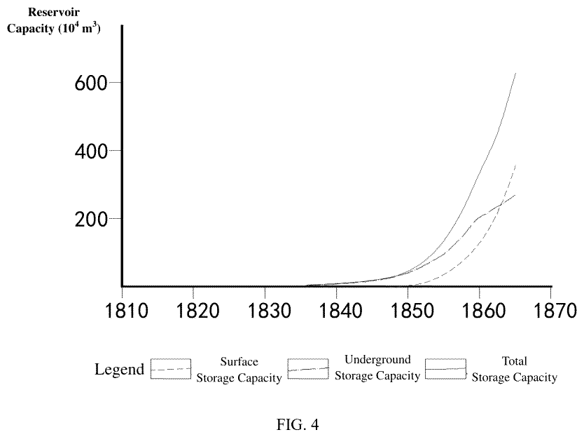

For underground storage capacity, the underground storage capacity should be the volume of all karst caves that can be filled with water in the underground karst body, which is determined by the product of the karst caves V_{cave} that can be explored and measured plus the remaining soluble rock volume V_{solution crack} after deducting the karst caves that can be explored and measured and the above karst specific yield V_{solution crack}, then the underground karst storage capacity is V=V_{cave}+V_{solution crack}. The calculated reservoir capacity curve is shown in Table 7 and FIG. 4.

TABLE 7
Storage Capacity Characteristics of Stone Forest Underground Reservoir

| | | | | | Dissolution | | Total Underground | |
|---|---|---|---|---|---|---|---|---|
| | Explored | Water-Bearing Rock | | | Storage Capacity | | Storage Capacity | |
| | Cumulative | Rock | | Deducted | | Dissolution | Total | Total |
| Water | Volume of | Mass | Karst | Net Volume | | Storage | Storage | Storage |
| Level | Karst Caves | Plane | Volume | of Karst | | Capacity | Capacity | Capacity |
| Elevation | (Ten | Area (Ten | (Ten | Caves (Ten | Specified | (Ten | (Ten | (Ten |
| m | Thousandm³) | Thousandm³) | Thousandm³) | Thousandm³) | Yield | Thousandm³) | Thousandm³) | Thousandm³) |
| 1866 | 0.07 | | 258.25 | 258.18 | 0.0654 | 16.88 | 16.95 | 287.23 |
| 1865 | 0.63 | 516.51 | 2266.45 | 2265.82 | 0.0285 | 64.57 | 65.2 | 270.28 |
| 1860 | 1.03 | 393.17 | 1711.92 | 1710.89 | 0.0622 | 106.42 | 107.45 | 205.08 |
| 1855 | 0.27 | 273.43 | 1151.54 | 1151.27 | 0.0495 | 56.99 | 57.26 | 97.63 |
| 1850 | 0.03 | 189.32 | 756.62 | 756.59 | 0.03 | 22.7 | 22.73 | 40.37 |
| 1845 | 0.03 | 119.39 | 482.87 | 482.84 | 0.0183 | 8.84 | 8.87 | 17.64 |
| 1840 | 0.16 | 74.75 | 272.89 | 272.73 | 0.0157 | 4.28 | 4.44 | 8.77 |
| 1835 | 0.26 | 35.07 | 102.49 | 102.23 | 0.022 | 2.25 | 2.51 | 4.33 |
| 1830 | 0.26 | 10.33 | 23.25 | 22.99 | 0.047 | 1.08 | 1.34 | 1.82 |
| 1825 | 0.39 | 0.67 | 0.46 | 0.07 | 0.0055 | 0.01 | 0.4 | 0.48 |
| 1820 | 0.08 | | 0 | 0 | | | 0.08 | 0.08 |
| Total | 3.21 | | 7026.74 | 7023.61 | 0.04 | 284.02 | 287.23 | |

### S6. Results

According to the analysis, the total storage capacity of stone forest underground reservoir is 6.4882 million m³ (including 3.7548 million m³ of ground storage capacity and 2.7334 million m³ of underground storage capacity). It is a fully annual regulation reservoir, and the underground storage capacity under different characteristic water levels can be obtained. The normal water storage level is 1864.58 m, the corresponding storage capacity is 5.9229 million m³, the beneficial storage capacity is 4.8718 million m³, the flood control storage capacity is 468,500 m³, and the dead water level is 1853.01 m (storage capacity is 722,400 m³).

### 1. Beneficial Technical Effects of the Technical Solution of the Invention

Through the trial water storage operation of stone forest underground reservoir, according to the upstream inflow (water) and outflow (water release) observation (water tank model simulation method), it is calculated that:

### (1) Inflow Analysis

According to the water storage test on Aug. 15, 2017, the measured upstream water flow during the same period was 0.21-1.79 m³/s, with an average flow of 0.455 m³/s. During the test water storage period, the water level rose from 1823.04 m to 1857.32 m, and the reservoir capacity was 2.1335 million m³. After deducting the surface storage capacity of 694,600 m³, the underground storage capacity was 1.4389 million m³, which was 103.2% of the calculated storage capacity of 1.4278 million m³ in the preliminary design stage. The calculated underground storage capacity was smaller when the storage capacity was below the elevation of 1856.37 m, and was 2%-4% larger when it was above this elevation. When the underground storage capacity was less than 1 million m³, the smaller the storage capacity, the greater the deviation, as shown in Table 8.

TABLE 8
Inflow Analysis Summary

| | | | | | | | Calculated | |
|---|---|---|---|---|---|---|---|---|
| | | | | | | | Underground | |
| | | | | | | | Storage Capacity | |
| | | | | | | | | Ratio of |
| | | | | | | | | Water |
| | | Inflow | | | Surface | Underground | Calculated | Storage |
| | Water | | | Total | Storage | Storage | Underground | Capacity |
| | Level | Flow | Inflow | Inflow | Capacity | Capacity | Storage | to Storage |
| Time | m | m³/s | 104 m³/s | 10⁴ m³/s | 10⁴ m³/s | 10⁴ m³/s | Capacity | Capacity |
| 8-15, 9:30:00 | 1823.04 | 1.31 | 0.944 | | | | 0.31 | 0 |
| 8-15, 11:30:00 | 1827.5 | | 0.677 | 1.62 | | 1.62 | 0.87 | 1.86 |
| 8-16, 9:30:00 | 1843.8 | | 3.384 | 5 | | 5 | 15.13 | 0.33 |
| 8-18, 10:00:00 | 1849.29 | 0.57 | 14.064 | 19.07 | 1.49 | 17.58 | 36.48 | 0.48 |
| 8-22, 10:00:00 | 1851.51 | 0.58 | 19.852 | 38.92 | 7.37 | 31.55 | 55.27 | 0.57 |
| 8-25, 16:00 | 1852.67 | 0.45 | 14.548 | 53.47 | 13.85 | 39.62 | 68.35 | 0.58 |
| 8-29, 16:00 | 1853.77 | 1.07 | 28.905 | 82.37 | 21.97 | 60.4 | 81.57 | 0.74 |
| 9-1, 16:00 | 1854.29 | 0.58 | 19.302 | 101.68 | 27.13 | 74.55 | 88.23 | 0.84 |
| 9-5, 16:00 | 1854.78 | 0.38 | 15.837 | 117.51 | 32.78 | 84.73 | 94.71 | 0.89 |
| 9-8, 16:00 | 1855.2 | 0.53 | 12.459 | 129.97 | 38 | 91.97 | 101.25 | 0.91 |
| 9-12, 16:00 | 1855.65 | 0.63 | 10.502 | 140.47 | 43.81 | 96.66 | 109.41 | 0.88 |
| 9-14, 16:00 | 1855.86 | 0.52 | 4.501 | 144.98 | 46.71 | 98.27 | 113.21 | 0.87 |
| 9-20, 16:00 | 1856.37 | 1.79 | 35.433 | 180.41 | 53.96 | 126.45 | 123.15 | 1.03 |
| 9-22, 16:00 | 1856.52 | 0.83 | 7.171 | 187.58 | 56.25 | 131.33 | 126.16 | 1.04 |
| 9-26, 16:00 | 1856.77 | 0.55 | 10.714 | 198.29 | 60.21 | 138.08 | 131.16 | 1.05 |
| 9-28, 16:00 | 1856.88 | 0.45 | 3.914 | 202.21 | 62 | 140.89 | 133.36 | 1.05 |
| 10-2, 16:00 | 1857.07 | 0.27 | 5.443 | 207.65 | 65.16 | 142.49 | 137.3 | 1.04 |
| 10-6, 16:00 | 1857.24 | 0.21 | 3.884 | 211.53 | 68.07 | 143.46 | 141.03 | 1.02 |
| 10-8, 16:00 | 1857.32 | | 1.814 | 213.35 | 69.46 | 143.89 | 142.78 | 1.01 |

### (2) Outflow Analysis

After the trial water storage, the gates were opened to release water on October 9. According to the outflow observations of the underground river dredging tunnel and the high culvert opening, the outflow volume and drop elevation increased until the outflow reached the natural base flow of the underground river (flow rate 0.11-0.09 m³/s) on November 29. After the water was released, the underground caves were explored on November 7. It was observed that there were still many cracks with a width of less than 3 mm and karst cracks with fillings, in which water was slowly seeping and released. From October 9 to November 29, the total outflow of dredged tunnels and high culverts was 2.1453 million m³ and 239,500 m³ respectively. After deducting the natural base flow of the underground river of 386,600 m³, the amount of water that can be supplied is 1.9983 million m³. After further deducting the surface storage capacity of 694,600 m³, the underground storage capacity is 1.3037 million m³, which is 91.0% of the storage capacity of 1.4278 million m³ calculated in the preliminary design stage. The observed cumulative storage capacity below the elevation of 1850 m is much larger than the calculated cumulative storage capacity due to the rapid water release speed. However, the outflow between the elevations above 1831.20 m is 40%-110% of the calculated underground storage capacity between the corresponding elevations. The results are shown in Table 9.

TABLE 9
Outflow Analysis Summary

| | | | | | | | | Total | | | |
|---|---|---|---|---|---|---|---|---|---|---|---|
| | | | | | | | Total Outflow | Storage | | | Ratio of |
| | | | | | | Surface | Between Each | Capacity | | Calculated | Total |
| | | | | | | Storage | Elevation | 10⁴ m³ | | Cumulative | Outflow to |
| | | | | | | Capacity | (Underground | From | From | Underground | Calculated |
| | Water | Outflow 10⁴ m³ | | | Total | at Each | Storage | High | Low | Storage | Cumulative |
| | Level | Water | Dredge | Base | Outflow | Elevation | Capacity) | to | to | Capacity10⁴ | Storage |
| Time | m | Culvert | Hole | Flow | 10⁴ m³ | 10⁴ m³ | 10⁴ m³ | Low | High | m³ | Capacity |
| October 9 | 1857.32 | 0 | 0 | 0 | 0 | | | | 130.37 | 142.78 | 0.91 |
| 9:50 | | | | | | | | | | | |
| 16:00 | 1857.15 | 1.39 | 3.00 | 0.26 | 4.13 | 2.94 | 1.19 | 1.19 | 129.2 | 139.06 | 0.93 |
| October 10 | 1856.39 | 4.15 | 9.01 | 0.71 | 12.44 | 11.93 | 0.51 | 1.71 | 128.7 | 123.55 | 1.04 |
| 10:00 | | | | | | | | | | | |
| October 11 | 1855.4 | 5.78 | 12.01 | 0.95 | 16.84 | 14.04 | 2.80 | 4.50 | 125.9 | 104.87 | 1.20 |
| 10:00 | | | | | | | | | | | |
| October 12 | 1854.33 | 5.70 | 12.01 | 0.95 | 16.76 | 12.76 | 4.00 | 8.51 | 121.9 | 88.76 | 1.37 |
| 10:00 | | | | | | | | | | | |
| October 14 | 1851.5 | 6.93 | 25.50 | 1.90 | 30.53 | 20.48 | 10.05 | 18.56 | 111.8 | 55.42 | 2.02 |
| 10:00 | | | | | | | | | | | |
| October 16 | 1831.2 | | 28.75 | 1.90 | 26.85 | 7.31 | 19.54 | 38.10 | 92.3 | 2.2 | 41.95 |
| 10:00 | | | | | | | | | | | |
| October 18 | 1824.75 | | 21.41 | 1.73 | 19.69 | | 19.69 | 57.78 | 72.6 | | |
| 10:00 | | | | | | | | | | | |
| October 20 | 1823 | | 10.30 | 1.56 | 8.74 | | 8.74 | 66.52 | 63.8 | | |
| 16:01 | | | | | | | | | | | |
| 2017 Oct. 30 | 1822.85 | | 32.66 | 6.30 | 26.36 | | 26.36 | 92.88 | 37.5 | | |
| 2017 Nov. 9 | 1822.73 | | 25.92 | 7.46 | 18.46 | | 18.46 | 111.34 | 19.0 | | |
| 2017 Nov. 20 | 1822.68 | | 19.18 | 7.46 | 11.72 | | 11.72 | 123.06 | 7.3 | | |
| 2017 Nov. 27 | 1822.62 | | 10.37 | 4.98 | 5.39 | | 5.39 | 128.45 | 1.9 | | |
| 2017 Nov. 30 | 1822.59 | | 4.41 | 2.49 | 1.92 | | 1.92 | 130.37 | 0.0 | | |
| 2017 Dec. 11 | 1822.59 | | | | | | | | | | |
| Total | | 23.95 | 214.53 | 38.66 | 199.83 | 69.46 | 130.37 | | | | |

### (3) Conclusion

The actual cavity volume of the karst cave can be calculated by deducting the surface volume from the actual inflow and outflow. The average volume karst rates below the elevation of 1857.34 m are 4.22% and 3.83%, respectively, which are 103% and 92% of the average value of 4.01% predicted in the preliminary design.

### 2. Compare the Following Methods Based on the Most Suitable Ones at Present

(1) The groundwater balance method was adopted. Based on the results of groundwater hydrological observation, the annual distribution analysis of the designed annual runoff (P=75%) of the underground reservoir was carried out. The runoff of 203,000 m³ in April, the driest period, was taken as the stable runoff. The stable runoff of stone forest underground reservoir is 2.436 million m³. The increased runoff from January to May in the dry season is 543,000 m³. The underground storage capacity will not be less than the sum of the stable runoff (2.436 million m³) and the seasonal increased runoff (547,000 m³) in the dry season (January to May). The underground storage capacity is 2.983 million m³.

(2) According to the Plotnikov method of groundwater runoff analysis, under natural conditions, the dynamic reserve of groundwater flowing through a certain water-passing section of the aquifer per unit time is 8.973 million m³/year. Under natural conditions, the regulating reserve (underground storage capacity) in the aquifer between the highest and lowest water levels is 2.0201 million m³/year. The static reserve that needs to be extracted by wells is approximately 30.15 million m³.

3. The underground storage capacity obtained based on hydrogeological parameters is 2.733 million m³, which is between 2.0201 million m³/year obtained by the Plotnikov method and 2.983 million m³/year obtained by the groundwater equilibrium method. Therefore, the underground storage capacity of the groundwater reservoir is determined to be 2.733 million m³.

Through the use of a variety of methods to analyze and demonstrate the underground reservoir capacity, we strive to objectively and accurately obtain relatively reliable results. The biggest highlight of the invention is that the spatial distribution function of unit water permeability, permeability coefficient, karst rate and water supply degree obtained by a large number of easily available hydrogeological tests is used to obtain the corresponding reservoir capacity curve, which is summarized through water storage operation inspection, and has a good guiding significance for the water storage, scheduling and flood control of underground reservoirs. Due to the decrease in precipitation in the reservoir area in recent years, in order to ensure water storage and drought resistance, the underground reservoir capacity between the 1857.34 m water storage level and the normal water storage level of 1864.58 m needs to be further reviewed and analyzed according to the water release observation during the water use process during operation.

The invention and the embodiments thereof are described hereinabove, and this description is not restrictive. What is shown in the drawings is only one of the embodiments of the invention, and the actual structure is not limited thereto. All in all, structural methods and embodiments similar to the technical solution without deviating from the purpose of the invention made by those of ordinary skill in the art without creative design shall all fall within the protection scope of the invention.

What is claimed is:

1. A calculation method of karst underground reservoir capacity curve based on hydrogeological parameters, including the following steps:
S1. hydrogeological exploration: conducting hydrogeological exploration of the target karst groundwater reservoir, including exploring the karst conditions of the dam site and reservoir area, and the distribution of karst groundwater;
S2. determination of karst underground water reservoirs scope: according to the hydrogeological conditions of the underground water reservoir basin of the target underground water reservoir, as well as the groundwater overflow elevation, groundwater springs and hydrogeological borehole data at the karst watershed, obtaining the groundwater level in the dry season and drawing the water level contour map accordingly to determine the boundary and bottom limit conditions of the underground water reservoir capacity;
S3. determination of water storage space: through ground geological mapping, calculating the karst fissure rate at multiple points, further surveying the karst caves between the highest water storage level and the lowest groundwater level, focusing on finding out the volume and elevation distribution of the karst caves that can be explored, and calculating the volume distribution of water storage rock bodies and karst caves that can be explored at different elevations above the lowest groundwater level;
S4. determination of relevant hydrogeological parameters based on hydrogeological tests: arranging multiple groups of exploration boreholes and pumping test holes in the catchment area of the target underground reservoir, and conducting a large number of borehole pressure/water injection tests to obtain hydrogeological parameters, including borehole void encounter rate and unit water permeability, and analyzing the drainage volume and specific yield of the cone of depression, as well as the specific yield of each elevation of the underground reservoir basin according to the depression curve obtained from the pumping test;
S5. underground reservoir capacity analysis: based on the borehole data, statistically analyzing the borehole line karst rate of each borehole, including the filled and unfilled types, and the unit water permeability according to the elevation distribution, and using the relevant section length for weighted average to calculate the borehole line karst rate and unit water permeability of each comprehensive borehole at each elevation;
according to the regression relationship between karst specific yield and comprehensive borehole line karst rate and unit water permeability μ=0.525n+0.088q, calculating the specific yield of each elevation involved in the underground reservoir capacity;
for underground storage capacity, the underground storage capacity should be the volume of all karst caves that can be filled with water in the underground karst body, which is determined by the product of the karst caves V_{cave} that can be explored and measured plus the remaining soluble rock volume V_{solution crack} after deducting the karst caves that can be explored and measured and the above karst specific yield V_{solution crack}, then the underground karst storage capacity is V=V_{cave}+V_{solution crack};
wherein V_{cave} is an unfilled karst cave of no less than 300 m³; V_{solution crack}=Vₜₒₜₐₗ−V_{cave}; V_{solution crack}=μv_{solution crack}.

2. The calculation method of karst underground reservoir capacity curve based on hydrogeological parameters of claim 1, wherein in step S4, the hydrogeological parameters obtained include the borehole line karst rate, the unit water permeability of the relevant rock mass, and the permeability coefficient.

3. The calculation method of karst underground reservoir capacity curve based on hydrogeological parameters of claim 2, wherein in step S4, the borehole line karst rate n/% is the ratio of the total length of the karst caves l/m seen in the borehole to the borehole footage L/m of this section:
$$n = \frac{l}{L} .$$

4. The calculation method of karst underground reservoir capacity curve based on hydrogeological parameters of claim 2, wherein in step S4, the unit water permeability q of the relevant rock mass is the amount of water injected Q/l/min under unit pressure P/MPa and unit test section length L/m:
$$q = \frac{Q}{P L} .$$

5. The calculation method of karst underground reservoir capacity curve based on hydrogeological parameters of claim 1, wherein in step S4, the permeability coefficient is obtained according to the Babushkin formula:
$$K = 0.3 \; 66 \frac{Q}{l s} 1 g \frac{0.66 l}{r}$$
in the formula: K is the permeability coefficient m/d, Q is the water inflow m³/d, l is the filter length (m), s is the drawdown (m), and r is the borehole radius.

6. The calculation method of karst underground reservoir capacity curve based on hydrogeological parameters of claim 1, wherein in step S4, the depression curve and drainage volume are calculated according to the Kusakin influence radius formula and the Weber specific yield formula:
Kusakin influence radius formula:
$$R = 2 s \sqrt{K H}$$
in the formula: s is the influence radius of the cone of depression (m), K is the permeability coefficient m/d, and His the thickness of the phreatic aquifer (m);
Weber formula:
$$R = 3 \sqrt{\frac{K H t}{μ}}$$
is transformed into:
$$μ = 9 \frac{K H t}{{R}^{2}}$$
in the formula: μ is the specific yield (%), t is the pumping time (d), and the specific yield calculated by using the influence radius of the Kusakin formula is 0-5%, with an average of 1.98%, which is difficult to correlate with the karst development at various elevations of the underground reservoir basin and the specific yield.

7. The calculation method of karst underground reservoir capacity curve based on hydrogeological parameters of claim 1, wherein in step S4, the method for calculating the drainage volume of the cone of depression includes the following steps:
(1) assuming: R is the maximum radius of the cone of depression (m), s is the maximum groundwater drawdown (m), rᵢ is the distance from the pumping hole axis (m), hᵢ is the water level (m) at rᵢ above the maximum drawdown elevation of the pumping hole, and K is the permeability coefficient m/d;
(2) according to the observation of the depression curve, rᵢ is related to hᵢ, K and s, and has an obvious exponential function relationship with hᵢ: assuming rᵢ to be a composite function of
$${h}_{i}^{α} ,$$
K^{β}, and s^{γ}, and performing trial calculations based on
$${h}_{i}^{α} ,$$
K^{β}, and s^{γ}, when α, β, and γ are 3.5, 0.46, and 1.02, respectively, the correlation coefficients between the calculated value and the measured value are 0.99, 0.95, and 0.97, respectively; the depression curve equation is:
$${r}_{i} = {{ξ}_{S}}^{1.02} {K}^{0.46} {h}_{i}^{3.5} ;$$
(3) in the formula, the coefficient ξ is adjusted according to the corresponding depression curve shape, and its value is 0.49-1.16; s and K are fixed values, so the comprehensive coefficient is set to be m=ξs^{1.02}K^{0.46};
(4) the volume of the body rotating around the h (y) axis of the depression curve is:
$$V = π {\int }_{0}^{{h}_{max}} {[ {( \frac{h}{m} )}^{3.5} ]}^{2} dh = \frac{π}{{m}^{7}} \frac{{h}^{8}}{8} {\vert}_{0}^{{h}_{max}} = \frac{π {h}_{max}^{8}}{8 {m}^{7}} .$$

8. The calculation method of karst underground reservoir capacity curve based on hydrogeological parameters of claim 1, wherein in step S4, the specific yield μ/% is determined by the ratio of the amount of water discharged by the cone of depression Q/m³ to the volume of the cone of depression V/m³, that is:
$$μ = \frac{Q}{V} .$$

9. The calculation method of karst underground reservoir capacity curve based on hydrogeological parameters of claim 1, wherein in step S4, the analysis of specific yield at each elevation of the underground reservoir basin includes the following steps:
(1) making full use of the relevant hydrogeological parameters obtained from the pumping test wells for regression analysis; for two relevant parameters, performing a univariate linear regression; if two or more parameters are involved, using a binary or multivariate regression analysis, specifically:
univariate linear regression uses a linear function formula: y=a+bx;
or an exponential function: y=10^{a}x^{b};
then
$$b = \frac{{\sum }_{i = 1}^{n} ( {x}_{i} - x ) ( {y}_{i} - y )}{{\sum }_{i = 1}^{n} {( {x}_{i} - \overline{x} )}^{2}} ,$$
a=y̅−bx̅, wherein: x̅ and y̅ are average values;
the correlation coefficient is:
$$r = \frac{{\sum }_{i = 1}^{n} ( {x}_{i} - x ) ( {y}_{i} - \overline{y} )}{{\sum }_{i = 1}^{n} {( {x}_{i} - x )}^{2} {\sum }_{i = 1}^{n} {( {y}_{i} - \overline{y} )}^{2}} ;$$
(2) assuming that the multiple linear regression is:
$$X = [ \begin{matrix}1 & {x}_{11} & ⋯ & {x}_{1 n} \\ 1 & {x}_{21} & ⋯ & {x}_{2 n} \\ ⋮ & ⋮ & ⋮ & ⋮ \\ 1 & {x}_{n 1} & ⋯ & {x}_{nn}\end{matrix} ]$$
$$Y = [ \begin{matrix}{y}_{1} \\ {y}_{2} \\ ⋮ \\ {y}_{n}\end{matrix} ]$$
then the regression coefficient \hat{β}=(X^{T}X)⁻¹X^{T}Y, and the linear function regression equation is:
$$y = {β}_{0} + {β}_{1} {x}_{1} + {β}_{2} {x}_{2} + ⋯ + {β}_{n} {x}_{n}$$
or, the exponential function regression equation is:
$$y = {10}^{{β}_{0}} {x}_{1}^{{β}_{1}} {x}_{2}^{{β}_{2}} … {x}_{n}^{{β}_{n}} ,$$
the multiple correlation coefficient is:
$${r}_{xy} = \frac{{\sum }_{i = 1}^{n} ( {y}_{i} - \overline{y} ) ( {\hat{y}}_{i} - \overline{y} )}{\sqrt{{\sum }_{i = 1}^{n} {( {y}_{i} - \overline{y} )}^{2} {( {\hat{y}}_{i} - \overline{y} )}^{2}}}$$
(3) the correlation coefficients r and r_{xy} are dimensionless values, and the range of r and r_{xy} is [−1,1]; when r=1, it is a completely positive correlation, and when r=−1, it is a completely negative correlation, wherein:
when |r| or |r_{xy}|>0.8, there is a high or significant correlation; when 0.5<|r| or |r_{xy}|≤0.8, there is a moderate correlation;
when 0.3<|r| or |r_{xy}|≤0.5, there is a low correlation; when |r| or |r_{xy}|≤0.3, there is no linear correlation;
(4) conducting a univariate linear correlation analysis between the borehole specific yield μₛ/% and the average borehole line karst rate n/%, permeability coefficient K/m/d, and unit water permeability q/lu in the corresponding drop depth according to the existing data;
(5) according to the existing data, the borehole karst specific yield and the comprehensive borehole line karst rate and unit water permeability are subjected to binary regression, and the relationship is:
$${μ}^{′} = - 4.18 + 1.32 n + 0.21 q ,$$
the multiple correlation coefficient is 0.89, which is highly correlated; after canceling the coefficient −4.18, analyzing the specific yield μ′ calculated by binary regression and the actual specific yield μₛ again by univariate regression to obtain the comparison expression:
$$μ = 0.525 n + 0.088 q ,$$
the correlation coefficient is 0.94, which is highly correlated.

10. The calculation method of karst underground reservoir capacity curve based on hydrogeological parameters of claim 1, wherein in step (4), in the borehole pressure/water injection test, the more developed the karst is, the greater its unit water permeability is; usually, for unfilled karst caves, it is impossible to conduct pressure/water injection tests when they have a certain width; during the pressure/water injection test, it is difficult to conduct statistics on the encounter rate of borehole caves for parts with smaller unit water permeability; therefore, to examine the karst development intensity and karst rate of a part, it is necessary to combine the karst rate and unit water permeability of the borehole line for simultaneous analysis; the filling material of the filled karst caves is mainly gravel clay or gravel sandy clay; through the physical property test of the filling soil, the average porosity of the filling soil is 0.51; therefore, the porosity of the filled karst caves is calculated as 51%; the comprehensive borehole line karst rate should be determined by adding 51% of the filled borehole line karst rate to 100% of the unfilled karst cave rate.
